(19) United States
(12) Patent Application Publication
(10) Pub. No.: US 2018/0091961 A1
(43) Pub. Date: Mar. 29, 2018

(54) SMART CASE

(71) Applicant: University of Massachusetts, Boston, MA (US)

(72) Inventors: Shaad Mahmud, New Bedford, MA (US); Honggang Wang, Shrewsbury, MA (US)

(21) Appl. No.: 15/597,534

(22) Filed: May 17, 2017

Related U.S. Application Data
(60) Provisional application No. 62/341,309, filed on May 25, 2016.

(51) Int. Cl. H04L 29/08; H04M 1/02; H04B 1/3888; H04R 1/02; A61B 5/0408; A61B 5/00

(52) U.S. Cl. CPC H04W 4/22; H04M 1/0202; H04B 1/3888; H04W 4/12; A61B 5/04085; A61B 5/6898; H04R 1/028

(57) ABSTRACT

The present invention relates to a cell phone case, particularly to a multifunction back cover for a cell phone case, wherein the back cover has an rechargeable power source connected to a microcontroller and an emergency notification button used to trigger a variety of functional responses in cooperation with the application mode of the cell phone, wherein said microcontroller is in electronic communication with said emergency notification button.

### CROSS-REFERENCE TO RELATED APPLICATIONS

The present application claims the benefit of U.S. Provisional Patent Application No. 62/341,309, filed on May 25, 2017, which is incorporated herein by reference.

### STATEMENT OF GOVERNMENTAL SUPPORT

This invention was made with government support under Grant number 1401711 awarded by the National Science Foundation. The government has certain rights in the invention.

### FIELD OF THE INVENTION

The present invention relates to a cell phone case, particularly to a multifunction back cover for a cell phone case, wherein the back cover has an rechargeable power source connected to a microcontroller and an emergency notification button used to trigger a variety of functional responses in cooperation with the application mode of the cell phone, wherein said microcontroller is in electronic communication with said emergency notification button.

### BACKGROUND OF THE INVENTION

Personal safety is an issue that concerns everyone. While one may feel safe at home with a security system, that feeling of protection may not extend far from the front door. While it is hard to imagine this happening, it is important to have a plan in case one needs to react quickly, whether one is spending an evening out on the town, field trip, or walking home from school. Only in the USA, each year on an average 293,066 people are victims of rape or sexual assault. That means one person is assaulted in every 107 seconds. 83% of girls, age group between 12-16, have experienced sexual assault in public places. A statistic shows that 1 out of every 3 American women have been a victim of physical assault. Moreover, experts claim that most of the physical assaults go unreported, hence these statistics likely show only a portion of the actual number of total victims. And these are statistics only from the USA, so that global figures are clearly significantly higher. A smart personal safety device can prevent a person from becoming a crime statistic.

The report found that 1 in 10 U.S. smartphone owners are victims of phone theft and 68 percent of the victims were unable to recover their device after the theft occurred. The reality is that whether a smartphone is white, black, or gold, it is now almost 30 times more valuable per ounce than a block of solid silver and almost as easy to convert discreetly into cash. So it is important to have a system to reduce the number of lost phones.

What is needed is a system of device that helps provide personal and property safety in emergency situations.

### SUMMARY OF THE INVENTION

The present invention relates to a cell phone case, particularly to a multifunction back cover for a cell phone case, wherein the back cover has an rechargeable power source connected to a microcontroller and an emergency notification button used to trigger a variety of functional responses in cooperation with the application mode of the cell phone, wherein said microcontroller is in electronic communication with said emergency notification button.

The described features, structures, or characteristics of the invention may be combined in any suitable manner in one or more embodiments. In the following description, numerous specific details are recited to provide a thorough understanding of embodiments of the invention. One skilled in the relevant art will recognize, however, that the invention may be practiced without one or more of the specific details, or with other methods, components, materials, and so forth. In other instances, well-known structures, materials, or operations are not shown or described in detail to avoid obscuring aspects of the invention.

In one embodiment, the invention comprises an emergency notification system comprising: a) a smartphone case, wherein the back of said smartphone case comprises an emergency notification button and a microcontroller, wherein said microcontroller is in electronic communication with said emergency notification button; b) a smartphone configured for insertion into said smartphone case, wherein said smartphone comprises an emergency notification system mobile application, wherein said microcontroller is in wireless communication with said emergency notification system mobile application; and c) a rechargeable power source connected to said microcontroller. In one embodiment, said emergency notification system mobile application comprises a plurality of preset emergency notification button patterns. In one embodiment, said preset emergency notification patterns are selected from the group consisting of emergency notification button activation frequencies and emergency notification button activation durations. In one embodiment, at least one of said preset emergency notification button patterns activates said emergency notification system mobile application to send at least one electronic message. In one embodiment, said one electronic message is selected from the group consisting of: current GPS location of said system, SMS, email, photo, and voice recording. In one embodiment, said emergency notification button is dome shaped. In one embodiment, said emergency notification button is encircled by a light emitting diode (LED) ring. In one embodiment, said light emitting diode ring indicates the electrical power available to said system. In one embodiment, said system further comprises a GPS module. In one embodiment, said system further comprises an energy harvesting module. In one embodiment, said system further comprises an volume booster. In one embodiment, said rechargeable power source is electronically connected to the charging port of said smartphone. In one embodiment, said smartphone case further comprises a first electrocardiographic sensor and a second electrocardiographic sensor.

In one embodiment, the invention comprises a smartphone case, wherein the back of said smartphone case comprises an emergency notification button and a microcontroller, wherein said microcontroller is in electronic communication with said emergency notification button. In one embodiment, said case further comprises a rechargeable power source. In one embodiment, said case further comprises a power cord connector. In one embodiment, said case further comprises a power button. In one embodiment, said emergency notification button is dome shaped. In one embodiment, said emergency notification button is encircled by a light emitting diode ring. In one embodiment, said case further comprises a first electrocardiographic sensor and a second electrocardiographic sensor. In one embodiment, said light emitting diode ring indicates the electrical power available to said case. In one embodiment, said case further comprises a GPS module. In one embodiment, said case further comprises an energy harvesting module. In one embodiment, said case further comprises a volume booster. In one embodiment, said rechargeable power source is electronically connected to the charging port of a smartphone.

Other objects, advantages, and novel features, and further scope of applicability of the present invention will be set forth in part in the detailed description to follow, taken in conjunction with the accompanying drawings, and in part will become apparent to those skilled in the art upon examination of the following, or may be learned by practice of the invention. The objects and advantages of the invention may be realized and attained by means of the instrumentalities and combinations particularly pointed out in the appended claims.

### Definitions

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, the term “case” is used to describe an enclosure for a device. In one embodiment, the case is a partial enclosure, which protects at least the outer edges of the device. In one embodiment, the case covers the back of the device and the sides. In one embodiment, the case contains additional functional modules. The Smart Case is an example of a smartphone case.

As used herein, the term “microcontroller” or “MCU” or “microcontroller unit” is used to describe a small computer (SoC) on a single integrated circuit containing a processor core, memory, and programmable input/output peripherals.

As used herein, the term “rechargeable power source” is used to describe a compact power source, such as a battery, which will power the Smart Case and will be used as external power source. In a preferred embodiment, said rechargable power source is a lithium ion battery.

As used herein, the term “preset emergency notification button patterns” is used to describe a panic button which can be activated with user defined pattern.

As used herein, the term “GPS location” is used to describe a location on the earth based upon the Global Positioning System (GPS), a space-based navigation system that provides location and time information in all weather conditions, anywhere on or near the Earth where there is an unobstructed line of sight to four or more GPS satellites, or based upon triangulation signals of cellular communication towers. If the panic button is activated preset emergency contact will be notified with the location of the user, also it can be tracked in real time through our website.

As used herein, the term “SMS” or “ Short Message Service” is used to describe text messaging service component of phone, Web, or mobile communication systems. It uses standardized communications protocols to allow fixed line or mobile phone devices to exchange short text messages. If the panic button is activated preset emergency contact may be notified with an emergency text (SOS).

As used herein, the term “email” or “e-mail” is used to describe messages distributed by electronic means from one computer user to one or more recipients via a network. If the panic button is activated preset emergency contact may be notified with an email. The email may also be stored upon the device and/or uploaded to a cloud service.

As used herein, the term “photo” or “ photograph” is used to describe a picture made using a camera, in which an image is focused onto film or other light-sensitive material and then made visible and permanent by chemical treatment, or stored digitally. If the panic button is activated preset emergency contact may be notified with a stream of photos. The photos may also be stored upon the device and/or uploaded to a cloud service.

As used herein, the term “voice recording” or “sound recording” is used to describe electrical or mechanical inscription and re-creation of sound waves, such as spoken voice, singing, instrumental music, or sound effects. If the panic button is activated preset emergency contact may be notified with a voice recording, also it will be stored in our server which can be retrieved for later use. In one embodiment, the voice recording may be 5-10 seconds in length.

As used herein, the term “panic button” refers to an interface button which may launches the emergency response functions according to a pre-determined pattern of touch or duration. It is directly connected to the microcontroller. This button may be activated in emergencies.

As used herein, the term “dome shaped” is used to describe a partially convex panic button which provides a direct interface to launch the emergency response in the Smart Case.

As used herein, the term “light emitting diode” or “LED” is used to describe a RGB (red, green, and blue) LED light encircles the panic button. In one embodiment, an LED may also affect the color of the panic button. The LED may be used for multiple purposes and indications.

As used herein, the term “GPS module” is used to describe a module that may be part of the Smart Case which provides location information for the Smart Case. The GPS module will be used for lost mode and or in case of emergency. It is powered with Smart Case power source so it can operate without the phone/device.

As used herein, the term “energy harvesting module” is used to describe a kinetic energy conversion module which will recharge the Smart Case power source, such as a Li-Ion battery, with motion.

As used herein, the term “volume booster” is used to describe an additional speaker attached to Smart Case, will be used to enhance the audio signal and may be used in conjunction with the emergency response or lost mode.

As used herein, the term “electrocardiographic sensor” is used to describe any of the two ECG sensors that collect information from the finger tip. The sensors are connected to the Smart Case microcontroller and the collected information may be displayed on a paired device, such as a mobile phone.

As used herein, the term “docking connection” is used to describe the connection between the Smart Case device and an interfacing device, such as a mobile phone. The Smart Case can be charged while user is charging their device. The docking connection may simultaneously charge both Smart Case and device, such as a phone. In one embodiment, the docking connection provides an avenue for electronic communication between the Smart Case and the docked device.

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1 shows a back view showing the separation of the case with a smartphone according to an embodiment of the present invention.

FIG. 2 shows a front view showing the separation of the case with a smartphone according to an embodiment of the present invention.

FIG. 3 shows a block diagram showing the internal circuit of a multifunction back cover for cell phone according to an embodiment of the present invention.

FIGS. 4A & B shows flowcharts showing the procedure of collecting vital signs (FIG. 4A) and flow chart of the panic buttons algorithm (FIG. 4B).

FIG. 5 shows an overall architecture of the full system.

### LIST OF REFERENCE NUMERALS

1 smartphone case (Smart Case)

2 lower part

3 RGB LED, ring shape LED

4 dome shape button

5 main control board, microprocessor

6 volume booster circuit

7 external battery pack

8 additional speaker

9 USB port

10 GPS module

11 Energy harvesting module

12 Bluetooth low energy

13 first ECG sensor

14 second ECG sensor

15 smartphone

### DESCRIPTION OF THE INVENTION

The present invention relates to a cell phone case, particularly to a multifunction back cover for a cell phone case, wherein the back cover has an rechargeable power source connected to a microcontroller and an emergency notification button used to trigger a variety of functional responses in cooperation with the application mode of the cell phone, wherein said microcontroller is in electronic communication with said emergency notification button.

Personal protection devices known in the art do not contemplate the herein disclosed novel features. One example is United States Patent Application Publication Number US 2016-0049982 A1 [1], describes a back cover for cell phone, particularly to a multifunction back cover for cell phone, wherein the back cover has an “entity” button providing at least one function, but which does not describe a microcontroller connected to the button, the circular LED or the electrodes described in the present invention.

Another example, “CLIKI,” is a smartphone case that has an integrated, and potentially removable button interface that links wirelessly (Bluetooth) to a smartphone and triggers particular pre-programmed responses using an application, but which does not describe a microcontroller connected to the button, the circular LED or the electrodes described in the present invention.

Another example, Cayote Case, is a smartphone case that has a “panic button” which activates a piercing 100+ decibel siren sounds and an emergency text message with GPS coordinates is sent to designated emergency contacts, but which does not describe a microcontroller connected to the button, the single button interface, the circular LED, or the electrodes described in the present invention.

Another example, SOS—Help Button (PRO) Phone Application, is a phone application which entails requests for emergency assistance after the launching of the application with the touch of the “button” application icon on the phone, but does not describe a physical button and does not comprise a separate device.

Another example, United States Patent Application Publication Number US 2015-0065082 A1 [2], describes a wearable accessory including a detachable/removable circuitry housing which may be activated to vibrate, communicate with at least one handheld device using a communication protocol, an image capturing device including a microphone for receiving audio and video signals at the wearable accessory, a USB port for charging the wearable accessory, a multiple-bit microprocessor configured and coupled for controlling functions of the wearable accessory, the microprocessor housed within the housing and a storage means for storing data representative of the signals, the storage means coupled with the microprocessor, but which does not describe a wireless device case, but rather describes a separate accessory that may be used separately to communicate emergency signals with the handheld device. The reference also does not describe a large button for the communication interface.

Another example, Snapfon ezTWO Senior Unlocked GSM Cell Phone with SOS Button, is a simple GMS cell phone which features a simple SOS button with one touch connection to emergency services and family. The company also offers a monitoring service. The emergency contact button is built into the phone rather than being a separate entity from the phone.

Another example, V.ALRT, is an accessory with a button that may be used in conjunction with a mobile phone and may be wirelessly linked to the device to trigger emergency response using a software application, but which does not describe a wireless device case.

Another example, U.S. Pat. No. 8,249,547 [3], describes an emergency alert accessory that pairs with a mobile phone, but does not describe a phone case with an emergency button.

Another example, WIPO application WO/2009/150268 [4], describes a device that may serve as an alarm and a geographical position identifier that includes a microprocessor, powered by a rechargeable battery, microprocessor associated with a GSM/GPRS communications module, but which does not describe a wireless device case.

Another example, European Patent EP2088567A3 [5], describes personal safety system comprising: a personal safety device and a base unit coupled over a local communications link, but which does not describe a wireless device case, but rather describes a separate accessory that may be used separately to communicate emergency signals with a handheld device.

Another example, United States Patent Application Publication Number US 2008-0303657 A1 [6], describes a system includes at least one alarm event detection device, and a central panel in communication with the personal safety device and the at least one alarm event detection device, but which does not describe a wireless device case.

Another example, United States Patent Application Publication Number US 2005-0249049 A1 [7], describes a watch-like personal security device that permits a person in distress to alert people adjacent the person that a problem has occurred, but which does not describe a wireless device case, but rather describes a separate stand alone device that may be used separately to communicate direct emergency signals.

Another example, United States Patent Application Publication Number US 2011-0103558 A1 [8], describes a “smart” safety lanyard capable of automatically contacting emergency response personnel, but which does not describe a wireless device case.

Another example, U.S. Pat. No. 7,567,174 [9], describes a device and method for determining and automatically transmitting a geographic location of a wireless alarm device during a potential emergency utilizing enhanced wireless communication and position location systems, but which does not describe a phone case.

### DETAILED DESCRIPTION OF THE INVENTION

In the following description, for purposes of explanation, specific details are set forth in order to provide an understanding of the invention. It will be apparent, however, to one skilled in the art that the invention may be practiced without these details.

One skilled in the art will recognize that embodiments of the present invention, some of which are described below, may be incorporated into a number of enterprise resource planning systems. Structures and devices shown in the figures are illustrative of exemplary embodiments of the invention and are meant to avoid obscuring the invention. Furthermore, connections between components and modules within the figures are not intended to be limited to direct connections. It can be modified or altered based on applications.

In one embodiment, the present invention is a smartphone case having an emergency notification button placed on the back of the case. In one embodiment, it is a dome shaped button for easy access and is powered by a lithium ion battery independent of the smartphone. With a single charge it can last for more than two months with the described system. To reduce false alarms, the emergency notification button sends a variety of electronic messages from a connected smartphone when the activation pattern of the button matches a preset pattern. It is fully customizable where the preset pattern comprises either an activation frequency (i.e. two taps to connect to friends and family and three taps to contact authorities) and/or a duration (e.g., how many seconds the button has been pushed). In one embodiment, once the emergency notification button is activated, one or more electronic messages are sent to predetermined electronic mail addresses. These electronic messages may comprise short message services (SMS) or email, and contain information such as global positioning system (GPS) location and/or a prerecorded voice message. Additionally, the smartphone case can be linked to a website such that the GPS location of the smartphone case can be tracked in real time and alternative voice recordings may be stored. In one embodiment, when activated, the emergency notification button provides information to a conventional microcontroller (e.g., rfDuino) comprising a built-in Bluetooth (BLE) 4.0 transceiver and 32-bit ARM Cortex processor (e.g., 15×15 mm in size and ultra-low power wireless microcontroller). Consequently, the microcontroller fits onto the case seamlessly.

In one embodiment, the invention contemplates a smartphone case incorporated with different functionality without making the case bulky. As most people have a smartphone these days, a multifunctional smartphone case could provide a means to add more features to enhance the functionality of the combined device, as the case is always attached to the phone. Additionally, there are a number of instances where people have lost their cell phone and unable to track it down. As smartphones can be quite valuable and contain vital personal information, there is a great desire to protect these devices and provide additional personal protection to the owner of the smartphone. The present invention aims is to solve these different problems with one single solution, a “Smart Case”.

Personal safety is an issue that concerns everyone. While one may feel safe at home with a security system, that feeling of protection may not extend far from one's front door. It is important to have a plan in case one needs to react quickly to an emergency, whether one spends an evening out on the town, field trip, or walking home from school. A smart personal safety device can provide options to extend personal safety. Although, there are many known devices and smartphone applications for personal safety. Most of them are wearable devices or alert systems based on the smartphone application.

For many, a lost or stolen cell phone requires contacting a service provider, shutting down an account and dealing with the consequences of the loss. Even if the device is connected to the cloud, and the cell phone is switched off, the owner will not be able to track it until it is connected to the Internet, but that is highly unlikely to happen. So there is value in recovering, rather than replacing, the handheld device of choice. The Smart Case of the current invention has its own GPS and an external battery to operate without turning on the cell phone. This can be a very attractive feature for cell phone insurance companies and consumers.

In the event the owner encounters cardiac problems or even the possibility of heart problems and as appropriate for one's situation, it is better to have an device that allows for self-diagnosis. While there are currently many handheld ECG devices available, choosing one can be daunting. The addition of another device to carry is a reduction in personal convenience. In case of emergency, it is very important to react fast and get access to the safety device. Having this function built into a smartphone accessory can provide a convenient solution to this scenario. In one embodiment of the present invention, the smart case has two ECG electrodes in front of the case, and simple finger contacts suffice for recording ECGs and also stores the data for later use. Based on the received signal it provides live suggestion and medical personnel can also retrieve the data's from cloud server.

Loss of power is a considerable concern to smartphone users. Loss of power at a crucial moment, with no means to recharge the device can also be a risk to personal safety. Power drainage is also one of the biggest challenges facing the mobile technology industry. One solution to this problem is have carry a portable battery charger, but these devices also run out and need to be recharged in the same way as other devices. In one embodiment, the present invention Smart Case has an energy harvesting module which generates power as it moves. Motion may provide an opportunity to recharge the power source for the device. Walking or other activity while the phone is in the user's pocket may create enough energy to recharge the attached phone up to 30% and may also recharge its external battery pack. The principle of working to convert kinetic energy into electrical energy, and there is an energy harvesting module to achieve this goal and place it with the smartphone case.

Additionally, some smartphone speakers might not be loud enough for some people. However, there are volume booster applications for mobile devices, but prolonged use of these apps may harm the device, provide a speed and power drain, and might even damage the internal speakers of the device. In some embodiments, the Smart Case of the present invention also includes a volume booster for smartphone which also improves the quality of the sound.

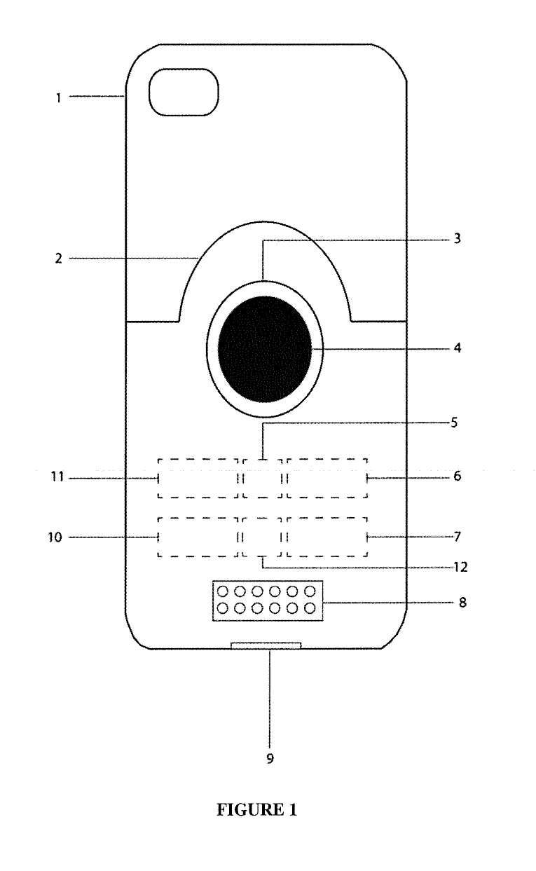

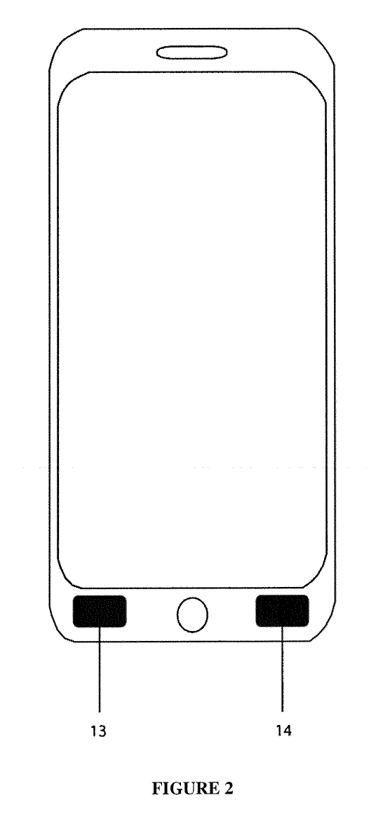

FIG. 1 shows a back of the smartphone case 1 which includes a dome shape button 4 encapsulated by an RGB led 3. The ring shape LED 5 will turn blinking red in case of low battery. The case can be removed or attached to the smartphone 15 by removing the lower part 2. The Smart Case 1 also includes, an additional GPS 10 to locate the device in case the phone has been lost and it is unable to connect to the Internet or the cell phone is turned off. As FIG. 2 refers, there are two sensors 13, 14 in front of the Smart Case 1 where the user just have to put their fingers on, the sensor will initiate measures ECG. The analog circuit for measuring ECG will be turned off to save power, only in case of skin contact it will be to wake up mode, this feature can also be controlled from the mobile app. The analog circuit is integrated into the main control board 5 and sends the collected information to the mobile phone via Bluetooth low energy 12 (BLE 4.0). Energy harvesting module 11 has also been installed on the Smart Case 1, the working principle is to convert kinetic energy into electrical energy. In one embodiment, a small inductor has been incorporated into a smartphone case 1. Module 11 provides at least 30% of an external battery pack 7 capacity. Module 11 is also connected to the main control board 5 to monitor the generated power. In one embodiment, to make the Smart Case 1 function independently the case comprises a lithium battery 7 of 1200-1500 mAh. This is charged by USB port 9 while the phone is charging and partially by the energy harvesting module 11. The Smart Case 1 has an additional speaker 8 with a volume booster 6 circuits which is used to amplify the audio signal or indication of the emergency button 4 has been pressed. The purpose of the speaker 8 is very simple, and is needed because the internal speaker of the smartphone might not be strong enough to produce clear and loud sound, but Smart Case 1 volume booster feature can solve this problem without buying an additional wireless speaker. Smart Case speaker 8 can be operated without the smartphone 15, as it is connected via Bluetooth 12. Smartphones have impressive capabilities, but a super-long battery life is elusive. Some people have turned to portable backup batteries to keep their phones charged, but one may still have to deal with finding a wall socket or universal serial bus (USB) port 9 to recharge the devices. So a self-charging battery 7 would solve these problems, and has the additional advantage that one would not need to carry an extra device.

The FCC reports that upwards of 48 percent of theft in major U.S. cities involves cell phones, and a survey found that just over 1 in 10 smartphone owners have been victims of phone theft [10]. One of the important features of the Smart Case 1 is to have its own GPS 10 module which runs independently from cell phone 15. Thus even if the cell phone is turned off, it is still traceable through Smart Case 1. Moreover, through use of a cloud server, the user can also store data in case loss of smartphone 15. So, not only it will help to retrieve the lost phone, also it will store data. Sometimes, the user may forget the last location of the phone, so the user can also trigger the buzzer through a cloud server to locate a lost phone.

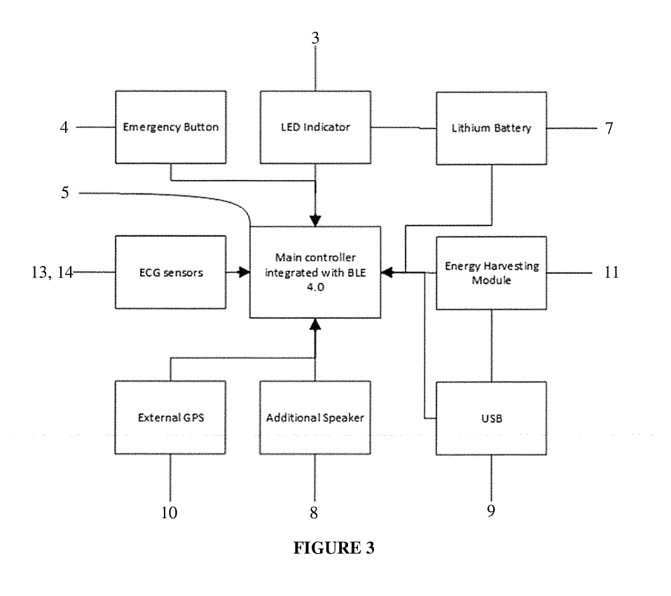

The full block diagram of Smart Case has been shown in FIG. 3. Panic button 4 is connected to RGB ring shape LED 3 and the microprocessor 5. To minimize power consumption, the LED 3 is not always on, only when the button 4 is pressed or the home screen of the phone is on. The LED 3 has been used for multipurpose, it indicates the activation of the emergency case, and it shows the electrical power of the external battery pack 7 also turns red in case of lost mode. The additional speaker 8 is coupled with audio control board 6 and with BLE 12. It works as a wireless speaker which can be used separately without installing it on smartphone 15. The charging circuit is coupled with energy harvesting module 11, USB port 9 and lithium battery 7. It also protects from any kind of electrical surge. The male USB port of the Smart Case 1 is attached to the smartphone and the female is left for external use. So, the user doesn't have to charge the Smart Case 1 separately, and instead it charges itself while charging the phone. It is very easy to monitor ECG on Smart Case 1, it is situated in a very convenient place where users can access comfortably and place their thumbs. Once skin contact is realized, it starts measuring the ECG and other vital information's. The mobile app not only stores and displays the data but can also help the user to analyze and find the best doctor near the user's current location. In some embodiments, the invention includes electrical modules which are connected to microcontroller 5, preferably Simblee. The fingertip sized Simblee is a slightly different kind of development board which comprises a built-in Bluetooth 4.0 12 transceiver and 32-bit ARM Cortex processor. The size and power consumption of Simblee make it a perfect component in certain embodiments of this invention. It is only 7×15mm in size and ultra-low power wireless microcontroller. Simblee can communicate with any Bluetooth 4.0 smartphone or tablet. More than that, it has more computing power than a general microprocessor, and it runs the same code as Arduino development boards. The entire unit can fit onto the case seamlessly. Lightweight and robust design make the present invention system a strong competitor among other security and safety devices.

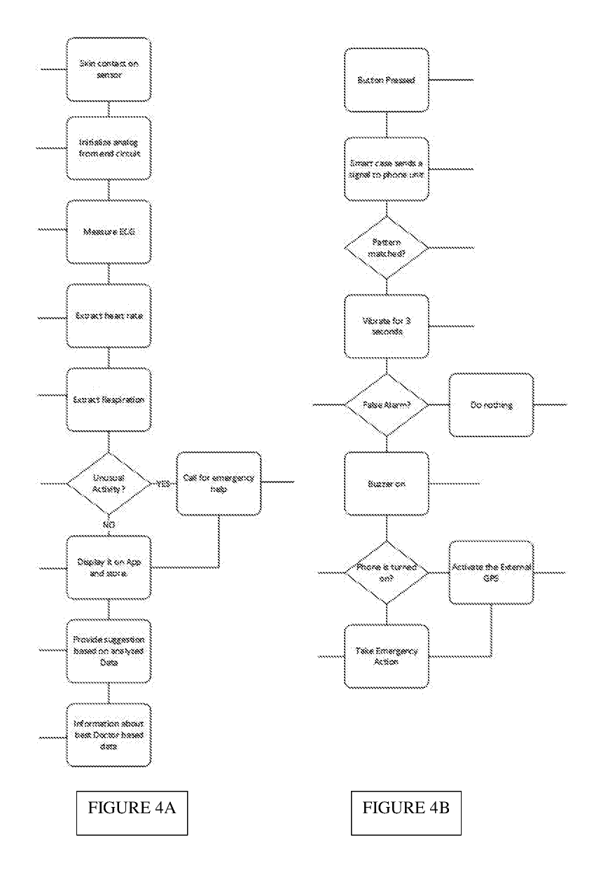

FIG. 4A shows a protocol to extract vital information from ECG sensors 13, 14 placed in front of the Smart Case 1. The user has to place the fingers on two sensors 13, 14, impedance change of these sensors will send to the microprocessor 5 to initiate the measurement of ECG. ECG circuit record the weak electrical signals produced by the heart and which spread throughout the body all the way to the skin. The Smart Case 1 picks up the signals by contact with the skin through interfaces called electrodes 13, 14. The process is non-invasive, harmless, and is expected to be risk-free. From ECG measurements, heart rate and respiration can be extracted, and since these data are collected in real time, additional analysis can be done. Vital information can be analyzed with a pre-set algorithm and based on that the suggestion will be given. For example, in case of unusual heart rate or respiration there are apps that can suggest what should be done and which doctor to see based on the current location. In addition to storing and viewing ECG records directly with the associated device, another method of uploading, analyzing, viewing, storing, and otherwise working with the ECG recordings may be used, such as a cloud-based system. The cloud server is connected to medical organizations and/or medical record services, to allow further analysis.

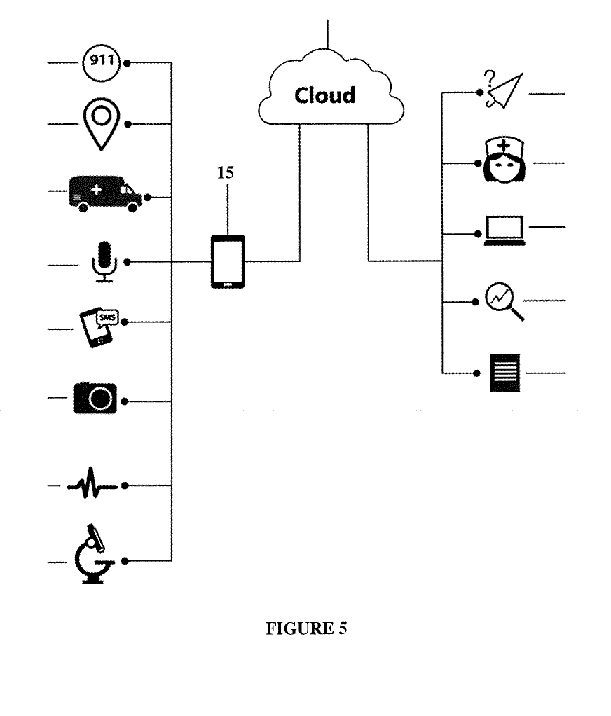

Turning now to FIG. 4B, the protocol is shown that controls how the panic button 4 works and sends the signal to smartphone 15. The panic button 4 is coupled with the main control board 5 and it requires a pattern to be activated. It is used to reduce the potential for false alarms, and the pattern can be set or changed through a mobile phone app. It is fully customizable for the users: for example, two taps to connect to friends and family, three taps to contact authorities. For example, it can be activated based on how many seconds the button has been pushed. The list of emergency contact includes police departments, campus security, 911 emergency centers, volunteer organizations, emergency medical service (EMS) providers, rescue departments, volunteers, employees, national security organizations including federal agencies, task forces and non-governmental agencies. Once it is activated the listed people will be notified with an SMS, email, photo, GPS location and five seconds voice recording (FIG. 5). Additionally, from a website or cloud server the device can be tracked in real time and the voice recordings will be stored in a server. This can be a subscription based service, in which each month Smart Case 1 users will subscribe based on their requirements. When the button 4 is pressed and confirmed, The LED 3 will indicate and cell phone will vibrate for three seconds to make sure it is not a false alarm. There can also be a gap of, for example, 5-10 seconds, before the emergency function is called. If the cell phone GPS location cannot be fetched, the Smart Case 1 will activate its own GPS 10 and send the data to users, which also can be tracked down through a cloud server. The cloud server is coupled with the Smart Case GPS 10 where the user can disable or enable lost mode.

The control features are preferably provided to the user as a mobile phone application. The user can edit and customize the features as he or she desires. The application has different tabs including an editable emergency contact list, patterns to activate the system and message that will be sent to the receiver. Further, the present invention could relate to the safety of women, children and senior citizens and extend to any individual needing emergency assistance. It could help support law enforcement agencies to act quickly to any distress signals. Overall, this could increase personal safety. Both an individual security and restorative ready gadget can give a quick approach to advice or help in a crisis circumstance. This may afford a straightforward and quick distinct option for informing guardians, relatives or crisis administrations about the user's present circumstances, including notification if the user is at risk of losing his or her life. The external GPS 10 of Smart Case 1 has a huge potential as one may not lose the phone even the phone is not in service or turned off. Additionally, the Smart Case 1 can monitor ECG (Electrocardiogram) by pressing two thumbs on the sensor. A user can record and check the daily activities with Smart Case. It also comes and extra feature of additionally battery 7 life through energy harvesting 11, provides our users a great opportunity to remain the working status of their smartphones by phone cases or appliances. As it were, clients will have the capacity to reduce charging times, and utilize the mobile phones longer and all the more viably. In addition, they do not need to remove the Smart Case or different assistants to charge their telephones.

The embodiments described above are only to exemplify the present invention, but not to limit the scope of the present invention. Therefore, any equivalent modification or variation according to the shapes, structures, features, or spirit disclosed by the present invention is to be also included within the scope of the present invention.

Important potential features of the Smart Case, which are present in some embodiments of the invention, include:
1. A smartphone case which has an emergency notification button used to trigger a variety of functional responses in cooperation with the application mode of the cell phone wherein the case is installed on the back of the cell phone.
2. A panic button, easy and convenient method which allows user to send and notify authorities in case emergency or distress. The system will send the current location of the Smart Case along with SMS, photo and voice recording.
3. Ring shape RGB LED will indicate electrical power of the battery and also on emergent case.
4. An additional GPS module to track down lost phone;
5. Health monitoring device, to track, record and analyze physical activity. If there is anomaly, it may notify the authorities.
6. Energy harvesting module to convert kinetic energy to electrical energy.
7. Volume booster to amplify the sound and notification of the emergency situation.
8. An extra lithium battery for backup and to run the Smart Case independently.
9. A self-charge mobile phone case.
10. The phone will vibrate 3 seconds to confirm the panic button has been pressed.
11. Smart Case will charge by itself while charging the phone.

Thus, specific descriptions and variations of the Smart Case have been disclosed. It should be apparent, however, to those skilled in the art that many more modifications besides those already described are possible without departing from the inventive concepts herein. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

Although the invention has been described with reference to these preferred embodiments, other embodiments can achieve the same results. Variations and modifications of the present invention will be obvious to those skilled in the art and it is intended to cover in the appended claims all such modifications and equivalents. The entire disclosures of all applications, patents, and publications cited above, and of the corresponding application are hereby incorporated by reference.

### REFERENCES

1. Chang, Y.-m. “Multifunction Back Cover for Cell Phone,” United States Patent Application Publication Number US 2016-0049982 A1, application Ser. No. 14/826,964, filed Aug. 14, 2015. (published Feb. 18, 2016).
2. Sehgal, A. “Personal Safety Device, System and a Method,” United States Patent Application Publication Number US 2015-0065082 A1, application Ser. No. 14/469,853, filed Aug. 27, 2014. (published Mar. 5, 2015).
3. Fellner, A. “Emergency Alert Device with Mobile Phone,” U.S. Pat. No. 8,249,547, application Ser. No. 13/134,757, filed Jun. 16, 2011. (issued Aug. 21, 2012).
4. Cerdán Torras, J. “Personal Safety Device,” WIPO PCT Patent Publication Number WO/2009/150268, Application PCT/ES2009/000316, filed Jun. 5, 2009. (published Dec. 27, 2009).
5. Clark, C. and Morgan, M. “Personal Safety System,” European Patent EP2088567A3, Application EP20090004558, filed Sep. 7, 2006. (issued Oct. 21, 2009).
6. Yuk, H. et al. “Life Safety Device for the Hearing Impaired,” United States Patent Application Publication Number US 2008-0303657 A1, application Ser. No. 11/759,800, filed Jun. 7, 2007. (published Dec. 22, 2008).
7. Jarrett, K. and Fitzgerald, P. “Personal Safety Device,” United States Patent Application Publication Number US 2005-0249049 A1, application Ser. No. 11/070,682, filed Mar. 1, 2005. (published Nov. 20, 2005).
8. Hooten, W. D. “Method and Apparatus for Activating a Communication Device Operably Connected to a Safety Lanyard,” United States Patent Application Publication Number US 2011-0103558 A1, application Ser. No. 12/917,132, filed Nov. 1, 2010, (published May 5, 2011).
9. Woodard, J. A. and Woodard, N. U. “Combination Alarm Device with Enhanced Wireless Notification and Position Location Features,” U.S. Pat. No. 7,567,174, application Ser. No. 11/320,011, filed Dec. 27, 2005. (issued Jul. 28, 2009).
10. FCC. (2012) “Announcement of New Initiatives to Combat Smartphone and Data Theft,” (Federal Communications Commission, Ed.).

We claim:

1. An emergency notification system comprising:
a) a smartphone case, wherein the back of said smartphone case comprises an emergency notification button and a microcontroller, wherein said microcontroller is in electronic communication with said emergency notification button;
b) a smartphone configured for insertion into said smartphone case, wherein said smartphone comprises an emergency notification system mobile application, wherein said microcontroller is in wireless communication with said emergency notification system mobile application; and
c) a rechargeable power source connected to said microcontroller.

2. The emergency notification system of claim 1, wherein said emergency notification system mobile application comprises a plurality of preset emergency notification button patterns.

3. The emergency notification system of claim 2, wherein said preset emergency notification patterns are selected from the group consisting of emergency notification button activation frequencies and emergency notification button activation durations.

4. The emergency notification system of claim 2, wherein at least one of said preset emergency notification button patterns activates said emergency notification system mobile application to send at least one electronic message.

5. The emergency notification system of claim 4, wherein said one electronic message is selected from the group consisting of: current GPS location of said system, SMS, email, photo, and voice recording.

6. The emergency notification system of claim 1, wherein said emergency notification button is dome shaped.

7. The emergency notification system of claim 1, wherein said emergency notification button is encircled by a light emitting diode ring.

8. The emergency notification system of claim 7, wherein said light emitting diode ring indicates the electrical power available to said system.

9. The emergency notification system of claim 1, wherein said system further comprises a GPS module.

10. The emergency notification system of claim 1, wherein said system further comprises an energy harvesting module.

11. The emergency notification system of claim 1, wherein said system further comprises an volume booster.

12. The emergency notification system of claim 1, wherein said rechargeable power source is electronically connected to the charging port of said smartphone.

13. The emergency notification system of claim 1, wherein said smartphone case further comprises a first electrocardiographic sensor and a second electrocardiographic sensor.

14. A smartphone case, wherein the back of said smartphone case comprises an emergency notification button and a microcontroller, wherein said microcontroller is in electronic communication with said emergency notification button.

15. The smartphone case of claim 14, further comprising a rechargeable power source.

16. The smartphone case of claim 14, further comprising a power cord connector.

17. The smartphone case of claim 14, further comprising a power button.

18. The smartphone case of claim 14, wherein said emergency notification button is dome shaped.

19. The smartphone case of claim 14, wherein said emergency notification button is encircled by a light emitting diode ring.

20. The smartphone case of claim 14, further comprising a first electrocardiographic sensor and a second electrocardiographic sensor.

21. The smartphone case of claim 14, further comprising a GPS module.

22. The smartphone case of claim 14, further comprising an energy harvesting module.

23. The smartphone case of claim 14, further comprising a volume booster.

24. The smartphone case of claim 15, further comprising a charging port.

25. The smartphone case of claim 24, further comprising a docking connection for a smartphone.
